(19) United States
(12) Patent Application Publication
(10) Pub. No.: US 2009/0270413 A1
(43) Pub. Date: Oct. 29, 2009

(54) DI-T-BUTYLPHENYL PIPERAZINES AS CALCIUM CHANNEL BLOCKERS

(76) Inventors: Robert Galemmo, JR., Vancouver (CA); Gabriel Hum, Langley (CA)

Correspondence Address: MORRISON & FOERSTER LLP, 12531 HIGH BLUFF DRIVE, SUITE 100, SAN DIEGO, CA, 92130-2040, US

(21) Appl. No.: 12/431,083

(22) Filed: Apr. 28, 2009

Related U.S. Application Data
(60) Provisional application No. 61/048,517, filed on Apr. 28, 2008.

(51) Int. Cl. A61K 31/497; A61P 25/00

(52) U.S. Cl. 514/253.08

(57) ABSTRACT

Methods and compounds effective in ameliorating conditions characterized by unwanted calcium channel activity, particularly unwanted N-type and/or T-type calcium channel activity are disclosed. Specifically, a series of compounds containing di-t-butyl phenyl piperazine derivatives of the general formula (1).

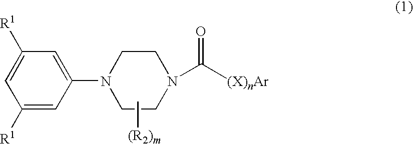

### RELATED APPLICATIONS

This application claims benefit of priority to U.S. Provisional Application Ser. No. 61/048,517 filed Apr. 28, 2008, the contents of which are incorporated herein by reference in its entirety.

### TECHNICAL FIELD

The invention relates to compounds useful in treating conditions associated with calcium channel function, and particularly conditions modulated by N-type and/or T-type calcium channel activity. More specifically, the invention concerns compounds containing di-t-butylphenyl piperazine derivatives that are useful in treatment of conditions such as pain, and other diseases or disorders of hyperexcitability.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Native calcium channels have been classified by their electrophysiological and pharmacological properties into T-, L-, N-, P/Q- and R-types (reviewed in Catterall, W., Annu Rev Cell Dev Biol (2000) 16: 521-555; Huguenard, J. R., Annu Rev Physiol (1996) 58: 329-348). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall (2000); Huguenard (1996)). T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed α_{1G}, α_{1H}, and α_{1I} (alternately called Cav 3.1, Cav 3.2 and Cav 3.3, respectively).

Calcium channels have been shown to mediate the development and maintenance of the neuronal sensitization and hyperexcitability processes associated with neuropathic pain, and provide attractive targets for the development of analgesic drugs (reviewed in Vanegas, H. & Schaible, H-G., Pain (2000) 85: 9-18). All of the high-threshold calcium channel types are expressed in the spinal cord, and the contributions of L-, N and P/Q-types in acute nociception are currently being investigated. In contrast, examination of the functional roles of these channels in more chronic pain conditions strongly indicates a pathophysiological role for the N-type channel (reviewed in Vanegas & Schaible (2000) supra).

Two examples of either FDA-approved or investigational drugs that act on N-type channels are gabapentin and ziconotide. Ziconotide (Prialt®; SNX-111) is a synthetic analgesic derived from the cone snail peptide Conus magus MVIIA that has been shown to reversibly block N-type calcium channels. In a variety of animal models, the selective block of N-type channels via intrathecal administration of ziconotide significantly depresses the formalin phase 2 response, thermal hyperalgesia, mechanical allodynia and post-surgical pain (Malmberg, A. B. & Yaksh, T. L., J Neurosci (1994) 14: 4882-4890; Bowersox, S. S. et al., J Pharmacol Exp Ther (1996) 279: 1243-1249; Sluka, K. A., J Pharmacol Exp Ther (1998) 287:232-237; Wang, Y-X. et al., Soc Neurosci Abstr (1998) 24: 1626).

Ziconotide has been evaluated in a number of clinical trials via intrathecal administration for the treatment of a variety of conditions including post-herpetic neuralgia, phantom limb syndrome, HIV-related neuropathic pain and intractable cancer pain (reviewed in Mathur, V. S., Seminars in Anesthesia, Perioperative Medicine and Pain (2000) 19: 67-75). In phase II and III clinical trials with patients unresponsive to intrathecal opiates, ziconotide has significantly reduced pain scores and in a number of specific instances resulted in relief after many years of continuous pain. Ziconotide is also being examined for the management of severe post-operative pain as well as for brain damage following stroke and severe head trauma (Heading, C., Curr Opin CPNS Investigational Drugs (1999) 1: 153-166). In two case studies ziconotide has been further examined for usefulness in the management of intractable spasticity following spinal cord injury in patients unresponsive to baclofen and morphine (Ridgeway, B. et al., Pain (2000) 85: 287-289). In one instance, ziconotide decreased the spasticity from the severe range to the mild to none range with few side effects. In another patient, ziconotide also reduced spasticity to the mild range although at the required dosage significant side effects including memory loss, confusion and sedation prevented continuation of the therapy.

Gabapentin, 1-(aminomethyl)cyclohexaneacetic acid (Neurontin™), is an anticonvulsant originally found to be active in a number of animal seizure models (Taylor, C. P. et al., Epilepsy Res (1998) 29: 233-249). Though not specific for N-type calcium channels, subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanical allodynia associated with postoperative pain (Taylor, et al. (1998); Cesena, R. M. & Calcutt, N. A., Neurosci Lett (1999) 262: 101-104; Field, M. J. et al., Pain (1999) 80: 391-398; Cheng, J-K., et al., Anesthesiology (2000) 92: 1126-1131; Nicholson, B., Acta Neurol Scand (2000) 101: 359-371).

While its mechanism of action is not completely understood, current evidence suggests that gabapentin does not directly interact with GABA receptors in many neuronal systems, but rather modulates the activity of high threshold calcium channels. Gabapentin has been shown to bind to the calcium channel α₂δ ancillary subunit, although it remains to be determined whether this interaction accounts for its therapeutic effects in neuropathic pain.

In humans, gabapentin exhibits clinically effective anti-hyperalgesic activity against a wide range of neuropathic pain conditions, Numerous open label case studies and three large double blind trials suggest gabapentin might be useful in the treatment of pain. Doses ranging from 300-2400 mg/day were studied in treating diabetic neuropathy (Backonja, M. et al., JAMA (1998) 280:1831-1836), postherpetic neuralgia (Rowbotham, M. et al., JAMA (1998) 280: 1837-1842), trigeminal neuralgia, migraine and pain associated with cancer and multiple sclerosis (Di Trapini, G. et al., Clin Ter (2000) 151: 145-148; Caraceni, A. et al., J Pain & Symp Manag (1999) 17: 441-445; Houtchens, M. K. et al., Multiple Sclerosis (1997) 3: 250-253; see also Magnus, L., Epilepsia (1999) 40 (Suppl 6): S66-S72; Laird, M. A. & Gidal, B. E., Annal Pharmacotherap (2000) 34: 802-807; Nicholson, B., Acta Neurol Scand (2000) 101: 359-371).

T-type calcium channels are involved in various medical conditions. In mice lacking the gene expressing the α_{1G} subunit, resistance to absence seizures was observed (Kim, C. et al., Mol Cell Neurosci (2001) 18 (2): 235-245). Other studies have also implicated the α_{1H} subunit in the development of epilepsy (Su, H. et al., J Neurosci (2002) 22: 3645-3655). There is strong evidence that some existing anticonvulsant drugs, such as ethosuximide, function through the blockade of T-type channels (Gomora, J. C. et al., Mol Pharmacol (2001) 60: 1121-1132).

Low voltage-activated calcium channels are highly expressed in tissues of the cardiovascular system. Mibefradil, a calcium channel blocker 10-30 fold selective for T-type over L-type channels, was approved for use in hypertension and angina. It was withdrawn from the market shortly after launch due to interactions with other drugs (Heady, T. N., et al., Jpn J Pharmacol. (2001) 85:339-350).

Growing evidence suggests T-type calcium channels are also involved in pain (see for example: US Patent Application No. 2003/086980; PCT Patent Application Nos. WO 03/007953 and WO 04/000311). Both mibefradil and ethosuximide have shown anti-hyperalgesic activity in the spinal nerve ligation model of neuropathic pain in rats (Dogrul, A., et al., Pain (2003) 105:159-168). In addition to cardiovascular disease, epilepsy (see also US Patent Application No. 2006/025397), and chronic and acute pain, T-type calcium channels have been implicated in diabetes (US Patent Application No. 2003/125269), certain types of cancer such as prostate cancer (PCT Patent Application Nos. WO 05/086971 and WO 05/77082), sleep disorders (US Patent Application No. 2006/003985), Parkinson's disease (US Patent Application No. 2003/087799); psychosis such as schizophrenia (US Patent Application No. 2003/087799), overactive bladder (Sui, G.-P., et al., British Journal of Urology International (2007) 99 (2): 436-441; see also US 2004/197825) and male birth control.

The present invention provides novel compounds having calcium channel activity, and which are active as inhibitors of N-type calcium channels in particular. These compounds are thus useful for treatment of disorders including pain and certain mood disorders, gastrointestinal disorders, genitourinary disorders, neurologic disorders and metabolic disorders.

### DISCLOSURE OF THE INVENTION

The invention relates to compounds useful in treating conditions modulated by calcium channel activity and in particular conditions modulated by N-type and/or T-type channel activity. The compounds of the invention are heterocyclic compounds with structural features that enhance the N-type and/or T-type calcium channel blocking activity of the compounds. Thus, in one aspect, the invention is directed to a method of treating conditions mediated by calcium channel activity by administering to patients in need of such treatment at least one compound of formula (1):

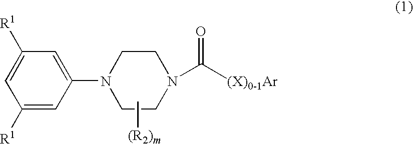

or a pharmaceutically acceptable salt or conjugate thereof, wherein

R₁ are independently C(CH₃)₃ or C(CF₃)₃;

each R₂ is independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′, —C(CH₃)₂CONR′₂, ═O, and ═NOR′, wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or R₂ may be one or more optionally substituted groups selected from alkyl (1-6C), alkenyl (2-6C), alkynyl (2-6C), heteroalkyl (2-6C), heteroalkenyl (2-6C), heteroalkynyl (2-6C);

m is 0-4 and n is 0-1;

X is alkylenyl (1-3C) or heteroalkylenyl (1-3C);

Ar is an optionally substituted aryl (6-10C) or heteroaryl (5-12 ring members);

wherein the optional substituents on Ar are independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′ and —C(CH₃)₂CONR′₂ wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12 ring members).

In another aspect, the invention is directed to pharmaceutical compositions containing these compounds and to the use of these compositions for treating conditions requiring modulation of calcium channel activity, and particularly N-type and/or T-type calcium channel activity. The invention is also directed to the use of these compounds for the preparation of medicaments for the treatment of conditions requiring modulation of calcium channel activity, and in particular N-type and/or T-type calcium channel activity.

### DETAILED DESCRIPTION

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-8C (alkyl) or 2-8C (alkenyl or alkynyl). In some embodiments, they contain 1-6C, 1-4C or 1-3C, 1-2C (alkyl); or 2-6C, 2-4C, or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In preferred embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In preferred embodiments, the heteroatom is O or N. For greater certainty, to the extent that alkyl is defined as 1-6C, then the corresponding heteroalkyl contains 2-6C, N, O, or S atoms such that the heteroalkyl contains at least one C atom and at least one heteroatom. Similarly, when alkyl is defined as 1-6C or 1-4C, the heteroform would be 2-6C or 2-4C respectively, wherein one C is replaced by O, N or S. Accordingly, when alkenyl or alkynyl is defined as 2-6C (or 2-4C), then the corresponding heteroform would also contain 2-6C, N, O, or S atoms (or 2-4) since the heteroalkenyl or heteroalkynyl contains at least one carbon atom and at least one heteroatom. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙOR, (CH₂)ₙCOR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the group contains at least one C and the size of the substituent is consistent with the definition of alkyl, alkenyl and alkynyl.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, 2-, 3- or 4-pyridyl, indolyl, 2- or 4-pyrimidyl, pyridazinyl, benzothiazolyl or benzimidazolyl. Even more particularly, such moiety is phenyl, pyridyl, or pyrimidyl and even more particularly, it is phenyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′, NR′SO₂R′, Si(CH₃)₃, CH₂CN, C(CH₃)₂CN, C(CH₃)₂CH₂OR′, C(CH₃)₂CO₂R′, C(CH₃)₂CONHR′ or C(CH₃)₂CONR′₂ wherein each R′ is independently H or an optionally substituted group selected from alkyl, alkenyl, alkynyl, heteroalkyl, heteroalkenyl, heteroalkynyl; or the substituent may be an optionally substituted group selected from alkyl, alkenyl, alkynyl, heteroalkyl, heteroalkenyl, heteroalkynyl.

Optional substituents on a non-aromatic group, are typically selected from the same list of substituents on aromatic or heteroaromatic groups and may further be selected from ═O and ═NOR′ where R′ is similarly defined.

Halo may be any halogen atom, especially F, Cl, Br, or I, and more particularly it is fluoro or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

R₁ is t-butyl or C(CF₃)₃ and in more particular embodiments, R₁ is t-butyl.

m is 0-4 and in more particular embodiments m is 0-2. In even more particular embodiments, m is 0 such that the central piperazine ring is unsubstituted. Similarly, n is 0 or 1 and in more particular embodiments, n is 0. When n is 1, X is an alkenyl or heteroalkenyl. In more particular embodiments, X contains 1-3 atom members selected from CH₂, CH₂CH₂, CH₂CH₂CH₂, CH₂O, or CH₂CH₂O. In even more particular embodiments, X is CH2O.

Ar may be furanyl, pyridinyl, thiadizolyl, phenyl, thiophenyl, pyrazolyl, thiazolyl, napthyl, naphthyridinyl, imidazolyl, isoxazolyl, pyrazolopyridinyl, oxazolyl, benzothiophenyl, quinolinyl or isothiazolyl. Optional substituents on Ar are as described above. In more particular embodiments, the optional substituents are selected from methyl, trifluoromethyl, phenyl, halo, methylsulfonyl, pivalamidyl, methoxy, t-butyl, isopropyl, (trifluoromethyl)phenyl, t-butylsulfonyl, propylthio, ethyl, 2-hydroxypropyl, 1,1,1,3,3,3-hexafluoro-2-hydroxypropyl, ethoxy, cyclopropyl, propyl, 2,2,2-trifluoroethoxy.

In some preferred embodiments, two or more of the particularly described groups are combined into one compound: it is often suitable to combine one of the specified embodiments of one feature as described above with a specified embodiment or embodiments of one or more other features as described above. For example, a specified embodiment includes X as benzhydryl and another specified embodiment has Ar as optionally substituted phenyl group. Thus one preferred embodiment combines both of these features together, i.e., X is benzhydryl in combination with both X representing optionally substituted phenyl. In some specific embodiments, R² is H and in other specific embodiments R² is methyl. Thus additional preferred embodiments include R² is H in combination with any of the preferred combinations set forth above; other preferred combinations include R² is methyl in combination with any of the preferred combinations set forth above.

The compounds of the invention may have ionizable groups so as to be capable of preparation as salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Compounds of formula (1) are also useful for the manufacture of a medicament useful to treat conditions characterized by undesired N-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

### MODES OF CARRYING OUT THE INVENTION

The compounds of formula (1) are useful in the methods of the invention and exert their desirable effects through their ability to modulate the activity of calcium channels, particularly the activity of N-type and/or T-type calcium channels. This makes them useful for treatment of certain conditions where modulation of N-type calcium channels is desired, including: chronic and acute pain; mood disorders such as anxiety, depression, and addiction; neurodegenerative disorders; hearing disorders; gastrointestinal disorders such as inflammatory bowel disease and irritable bowel syndrome; genitourinary disorders such as urinary incontinence, interstitial colitis and sexual dysfunction; neuroprotection such as cerebral ischemia, stroke and traumatic brain injury; and metabolic disorders such as diabetes and obesity. Certain conditions where modulation of T-type calcium channels is desired includes: cardiovascular disease; epilepsy; diabetes; certain types of cancer such as prostate cancer; pain, including both chronic and acute pain; sleep disorders; Parkinson's disease; psychosis such as schizophrenia; overactive bladder and male birth control.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

Anxiety as used herein includes but is not limited to the following conditions: generalized anxiety disorder, social anxiety disorder, panic disorder, obsessive-compulsive disorder, and post-traumatic stress syndrome. Addiction includes but is not limited to dependence, withdrawal and/or relapse of cocaine, opioid, alcohol and nicotine.

Neurodegenerative disorders as used herein include Parkinson's disease, Alzheimer's disease, multiple sclerosis, neuropathies, Huntington's disease, presbycusis and amyotrophic lateral sclerosis (ALS).

Cardiovascular disease as used herein includes but is not limited to hypertension, pulmonary hypertension, arrhythmia (such as atrial fibrillation and ventricular fibrillation), congestive heart failure, and angina pectoris.

Epilepsy as used herein includes but is not limited to partial seizures such as temporal lobe epilepsy, absence seizures, generalized seizures, and tonic/clonic seizures.

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

It is known that calcium channel activity is involved in a multiplicity of disorders, and particular types of channels are associated with particular conditions. The association of N-type and/or T-type channels in conditions associated with neural transmission would indicate that compounds of the invention which target N-type and/or T-type receptors are most useful in these conditions. Many of the members of the genus of compounds of formula (1) exhibit high affinity for N-type and/or T-type channels. Thus, as described below, they are screened for their ability to interact with N-type and/or T-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

There are three distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

A third type of inhibition is designated “resting channel block”. Resting channel block is the inhibition of the channel that occurs in the absence of membrane depolarization, that would normally lead to opening or inactivation. For example, resting channel blockers would diminish the peak current amplitude during the very first depolarization after drug application without additional inhibition during the depolarization.

In order to be maximally useful in treatment, it is also helpful to assess the side reactions which might occur. Thus, in addition to being able to modulate a particular calcium channel, it is desirable that the compound has very low activity with respect to the hERG K⁺ channel which is expressed in the heart. Compounds that block this channel with high potency may cause reactions which are fatal. Thus, for a compound that modulates the calcium channel, it should also be shown that the hERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Examples 3 and 4.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, e.g., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to activate or block the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to block the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Accordingly, a library of compounds of formula (1) can be used to identify a compound having a desired combination of activities that includes activity against at least one type of calcium channel. For example, the library can be used to identify a compound having a suitable level of activity on N-type calcium channels while having minimal activity on HERG K+ channels.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) or (2) may be used alone, as mixtures of two or more compounds of formula (1) and/or (2) or in combination with other pharmaceuticals. An example of other potential pharmaceuticals to combine with the compounds of formula (1) would include pharmaceuticals for the treatment of the same indication but having a different mechanism of action from N-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

The compounds of the invention may be prepared and used as pharmaceutical compositions comprising an effective amount of at least one compound of formula (1) admixed with a pharmaceutically acceptable carrier or excipient, as is well known in the art. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.01-15 mg/kg, preferably 0.1-10 mg/kg. However, dosage levels are highly dependent on the nature of the condition, drug efficacy, the condition of the patient, the judgment of the practitioner, and the frequency and mode of administration. Optimization of the dosage for a particular subject is within the ordinary level of skill in the art.

Synthesis of the Invention Compounds

A variety of synthetic methods familiar to those skilled in the art of Organic Chemistry may be employed in the preparation of compounds of Formula 1. In this discussion it will be recognized by a skilled practitioner that a sequence proposed for one series of compounds may require minor modifications, such as a re-ordering of synthetic steps, the use of different reaction conditions or reagents, or the selection of an alternative protecting group scheme, to be effective in producing the desired analog of Formula 1. References describing the use and limitations of protecting groups can be found in Greene and Wuts, Protective Groups in Organic Synthesis, Wiley-Interscience. References describing synthetic transformations can be found in Larock, Comprehensive Organic Transformations, Wiley-VCH. It is understood, however, that these compendia contain only some of the protecting groups and synthetic reactions that are available to one skilled in the art to prepare the compounds of Formula 1.

The following examples are offered to illustrate but not to limit the invention.

### Example 1

### Synthesis of 1-(3,5-di-tert-butylphenyl)piperazine

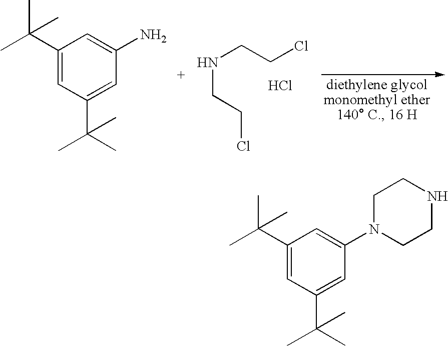

3,5-di-tert-Butylaniline (9 g, 43.8 mmol, 1 eq.) was combined with bis(2-chloroethyl)amine hydrochloride (7.82 g, 43.8 mmol, 1 eq.) in diethylene glycol monomethyl ether (11.7 mL). The reaction was heated at 140° C. for 16 hours. The homogeneous reaction was cooled and dissolved into an aqueous solution of NaOH (2N, 50 mL). The aqueous solution was extracted with ethyl acetate (4×100 mL). The pooled organic extracts were dried (Na₂SO₄) and concentrated to yield a gummy solid. The resulting crude was dissolved in diethyl ether (100 mL) and acidified with HCl in diethyl ether (2M, 100 mL). The excess solvent was removed in vacuo. The crude HCl salt was stirred in 1:1 hexanes:diethyl ether (300 mL) for 2 hours and then filtered by vacuum filtration to yield a beige precipitate. The beige precipitate was dissolved in an aqueous NaOH solution (2N, 50 mL) which was extracted with EtOAc (4×150 mL). The pooled organic fractions were dried (Na₂SO₄) and concentrated under reduced pressure. The resulting crude was purified by silica gel chromatography on a Biotage flash chromatography system (R_{f} 0.4 in 18:1:1 DCM:MeOH:Et₃N) to yield the desired product as a tan solid (6.4 g, 53% yield).

### Example 2

### Synthesis of Amides in Library Format

Compounds 1 through 27, 28 through 44, and 45 through 52 (Table 1) were prepared using parallel synthesis techniques and purified via mass direct preparative reverse phase HPLC. The amide couplings were accomplished via Method 1 or 2. No advantage in terms of yield and/or purity was observed in using either procedure.

Method 1: Amide Coupling Via Mixed Anhydride Formation

General Procedure

Carboxylic acid (375 μmol, 1.25 eq.) was suspended in THF (3 mL) and cooled to 0° C. N-methyl morpholine (420 μmol, 1.4 eq.) and i-butyl chloroformate (390 μmol, 1.3 eq.) were added and the reaction stirred at 0° C. for 2 hours. After 2 hours, 1-(3,5-di-tert-butylphenyl)piperazine (300 μmol, 1 eq) in THF (1 mL) was added. The reaction was stirred at room temperature for 72 hours. After 72 hours, the reactions were scavenged with silica bound diamine (1 eq.) and silica bound isocyanate (1 eq.) for 20 hours. Upon completion of the scavenging, the crude reactions were filtered and dried down. The desired product was purified by mass directed preparative reverse phase HPLC.

### Example

### Synthesis of N-(4-4(3,5-di-tert-butylphenyl)piperazine-1-carbonyl)pyridine-2-yl)pivalamide (Compound 22)

2-Pivalamidoisonicotinic acid (83.34 mg, 375 μmol, 1.25 eq.) was dissolved in THF (3 mL) and reacted with N-methyl morpholine (46.18 μL/42.48 mg, 420 μmol, 1.4 eq.) and i-butyl chloroformate (50.59 μL/53.27 mg, 390 μmol, 1.3 eq.) at 0° C. The reaction was stirred at 0° C. for 2 hours. After 2 hours, 1-(3,5-di-tert-butylphenyl)piperazine (300 μmol, 1 eq.) in THF (1 mL) was added and the reaction stirred at room temperature for 72 hours. The reaction was scavenged with silica bound diamine (1 eq.) and silica bound isocyanate (1 eq.) for 24 hours. The crude reaction was filtered and the silica washed with THF (4 mL). The crude was dried down. The desired product was purified by mass directed preparative reverse phase HPLC.

Method 2: Amide Coupling Via HATU Coupling

General Procedure:

Carboxylic acid (250 μmol, 1.25 eq.) was combined with N,N,N′,N′-tetramethyl-O-(7-azabenzotriazol-1-yl)uranium hexafluorophosphate (300 μmol, 1.5 eq.), triethylamine (800 μmol, 4 eq.), and 1-(3,5-di-tert-butylphenyl)piperazine (200 μmol, 1 eq.) in dichloromethane (4 mL). The reaction was stirred at room temperature for 72 hours. Upon completion of the reaction, silica bound scavenging reagents Si-isocyanate and Si-carbonate (1 eq. of each) were added. The reactions were scavenged for 20 hours after which they were filtered. The silica was washed with dichloromethane (4 mL). The crude products were dried. The desired products were purified by mass directed preparative reverse phase HPLC.

### Example

### Synthesis of (4-(3,5-di-tert-butylphenyl)piperazin-1-yl)(5-methylthiophen-2-yl)methanone (Compound 28)

5-Methylthiophene-2-carboxylic acid (35.54 mg, 250 μmol, 1 eq.) was combined with N,N,N′,N′-tetramethyl-O-(7-azabenzotriazol-1-yl)uranium hexafluorophosphate (114.07 mg, 300 μmol, 1.5 eq.), triethylamine (111.29 μL/80.80 mg, 800 μmol, 4 eq.), and 1-(3,5-di-tert-butylphenyl)piperazine (200 μmol, 1 eq.) in dichloromethane (4 mL). The reaction was stirred at room temperature for 72 hours. After 72 hours, the reactions were scavenged with silica bound isocyanate (1 eq.) and silica bound carbonate (1 eq.) for 20 hours. The crude reactions were filtered and the silica based scavenging reagents were washed with dichloromethane (4 mL). The crude reactions were dried and the final products purified by preparative reverse phase HPLC.

### Example 3

### Synthesized Compounds

Following the general procedures set forth above, the following compounds listed in Table 1 below were prepared.

TABLE 1

| Cmpd | | |
|---|---|---|
| No. | Name | Structure |
| 1 | (4-(3,5-di-tert-butylphenyl)piperazin-1- yl)(5-methyl-2-(trifluoromethyl)furan- 3-yl)methanone | 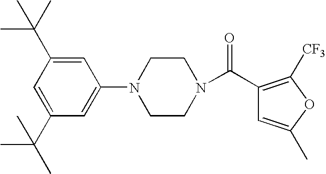 |
| 2 | (4-(3,5-di-tert-butylphenyl)piperazin-1- yl)(pyridin-4-yl)methanone | 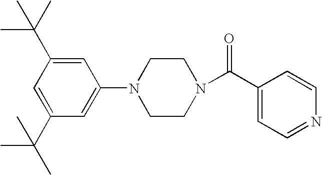 |
| 3 | (4-(3,5-di-tert-butylphenyl)piperazin-1- yl)(4-phenyl-1,2,3-thiadiazol-5- yl)methanone | 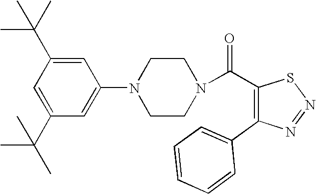 |
| 4 | (4-(3,5-di-tert-butylphenyl)piperazin-1- yl)(4-fluorophenyl)methanone | 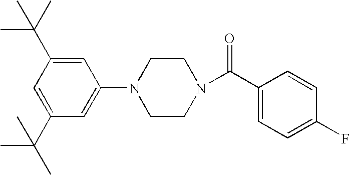 |
| 5 | (2-chloro-4-(methylsulfonyl)phenyl)(4- (3,5-di-tert-butylphenyl)piperazin-1- yl)methanone | 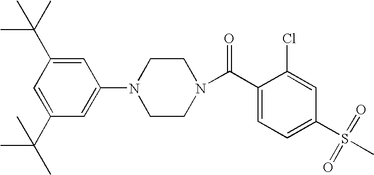 |
| 6 | (3-chloro-4-(methylsulfonyl)thiophen- 2-yl)(4-(3,5-di-tert- butylphenyl)piperazin-1-yl)methanone | 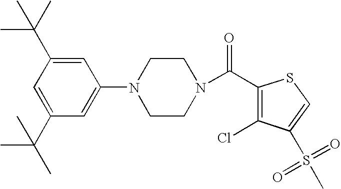 |
| 7 | N-(3-(4-(3,5-di-tert- butylphenyl)piperazine-1- carbonyl)pyridin-4-yl)pivalamide | 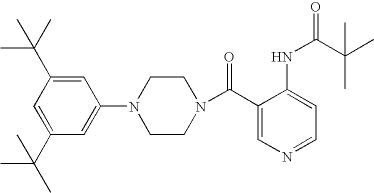 |
| 8 | N-(3-(4-(3,5-di-tert- butylphenyl)piperazine-1- carbonyl)pyridin-2-yl)pivalamide | 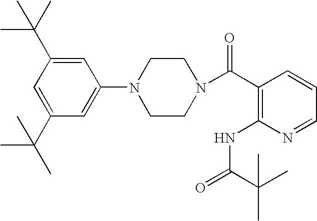 |
| 9 | (5-chloro-2-methoxyphenyl)(4-(3,5-di- tert-butylphenyl)piperazin-1- yl)methanone | 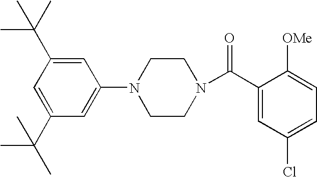 |
| 10 | (1-tert-butyl-3-methyl-1H-pyrazol-5- yl)(4-(3,5-di-tert- butylphenyl)piperazin-1-yl)methanone | 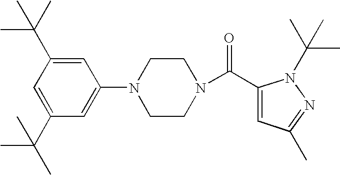 |
| 11 | (3-tert-butyl-1-methyl-1H-pyrazol-5- yl)(4-(3,5-di-tert- butylphenyl)piperazin-1-yl)methanone | 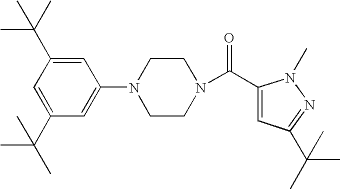 |
| 12 | (4-(3,5-di-tert-butylphenyl)piperazin-1- yl)(3-fluoro-5- (trifluoromethyl)phenyl)methanone | 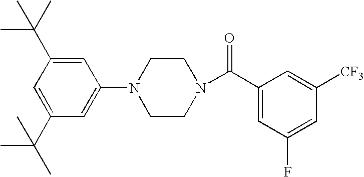 |
| 13 | 1-(4-(3,5-di-tert-butylphenyl)piperazin- 1-yl)-2-(4-fluorophenoxy)ethanone | 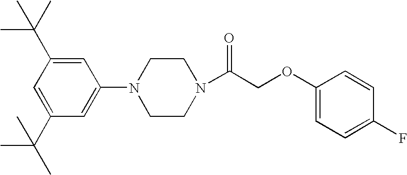 |
| 14 | 1-(4-(3,5-di-tert-butylphenyl)piperazin- 1-yl)-2-(2,4-dimethylphenoxy)ethanone | 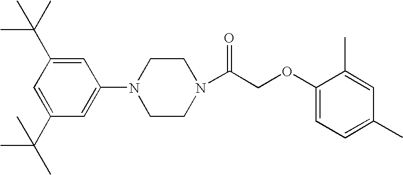 |
| 15 | (4-(3,5-di-tert-butylphenyl)piperazin-1- yl)(2,4-dimethylphenyl)methanone | 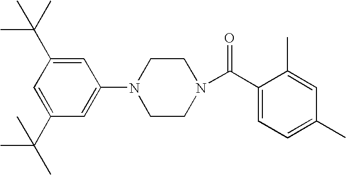 |
| 16 | (4-(3,5-di-tert-butylphenyl)piperazin-1- yl)(4-fluoro-3- (trifluoromethyl)phenyl)methanone | 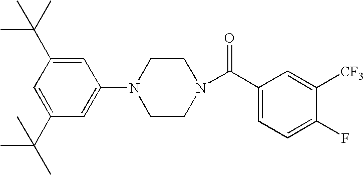 |
| 17 | (4-(3,5-di-tert-butylphenyl)piperazin-1- yl)(4-isopropylphenyl)methanone | 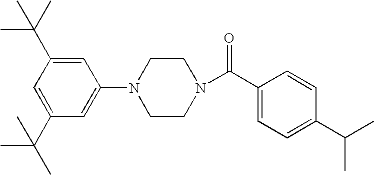 |
| 18 | (4-(3,5-di-tert-butylphenyl)piperazin-1- yl)(2-(3- (trifluoromethyl)phenyl)thiazol-4- yl)methanone | 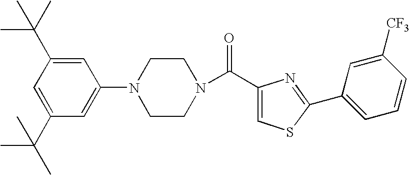 |
| 19 | (4-(3,5-di-tert-butylphenyl)piperazin-1- yl)(2-(trifluoromethyl)-1,8- naphthyridin-2-yl)methanone | 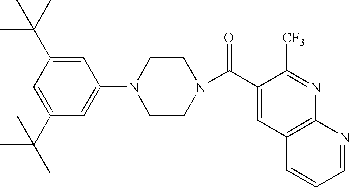 |
| 20 | (4-(3,5-di-tert-butylphenyl)piperazin-1- yl)(1-(2-(trifluoromethyl)phenyl)-1H- imidazol-2-yl)methanone | 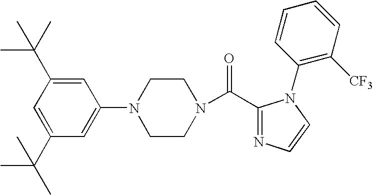 |
| 21 | (5-(tert-butylsulfonyl)thiophen-2-yl)(4- (3,5-di-tert-butylphenyl)piperazin-1- yl)methanone | 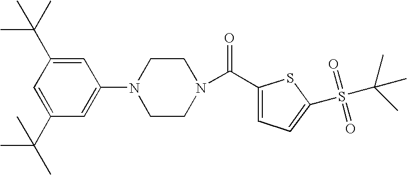 |
| 22 | N-(4-(4-(3,5-di-tert- butylphenyl)piperazine-1- carbonyl)pyridin-2-yl)pivalamide | 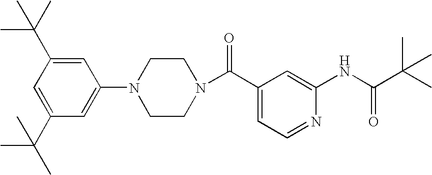 |
| 23 | (4-(3,5-di-tert-butylphenyl)piperazin-1- yl)(5-isopropylthiophen-3- yl)methanone | 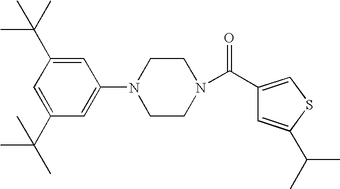 |
| 24 | (4-(3,5-di-tert-butylphenyl)piperazin-1- yl)(3-isopropylisoxazol-5-yl)methanone | 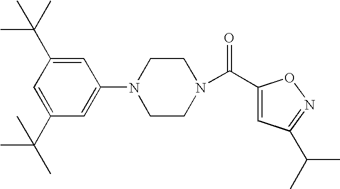 |
| 25 | (2-chloro-5-(methylsulfonyl)phenyl)(4- (3,5-di-tert-butylphenyl)piperazin-1- yl)methanone | 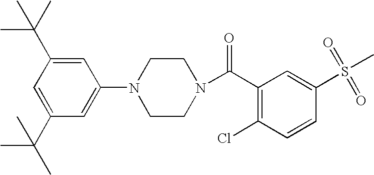 |
| 26 | (4-(3,5-di-tert-butylphenyl)piperazin-1- yl)(2-methyl-5- (methylsulfonyl)phenyl)methanone | 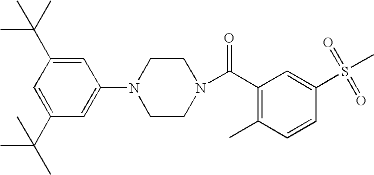 |
| 27 | (4-(3,5-di-tert-butylphenyl)piperazin-1- yl)(1,6-diisopropyl-1H-pyrazolo[3,4- b]pyridin-4-yl)methanone | 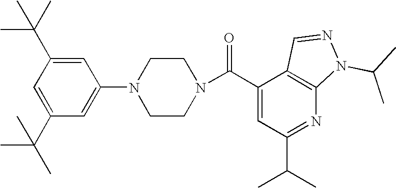 |
| 28 | (4-(3,5-di-tert-butylphenyl)piperazin-1- yl)(5-methylthiophen-2-yl)methanone | 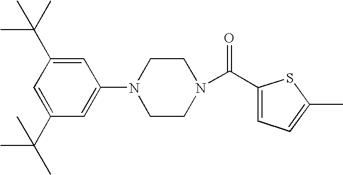 |
| 29 | (4-(3,5-di-tert-butylphenyl)piperazin-1- yl)(3-methylthiophen-2-yl)methanone | 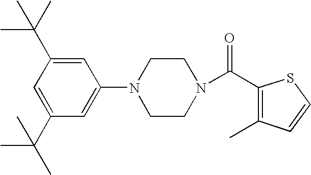 |
| 30 | (4-(3,5-di-tert-butylphenyl)piperazin-1- yl)(2-methyl-6- (trifluoromethyl)pyridin-3- yl)methanone | 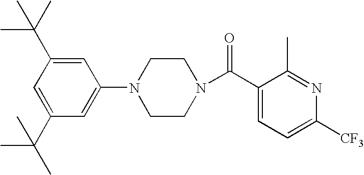 |
| 31 | (4-(3,5-di-tert-butylphenyl)piperazin-1- yl)(2-(propylthio)pyridin-3- yl)methanone | 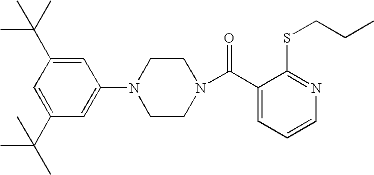 |
| 32 | (4-(3,5-di-tert-butylphenyl)piperazin-1- yl)(2,4-dimethylthiazol-5-yl)methanone | 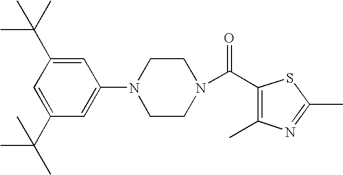 |
| 33 | (4-(3,5-di-tert-butylphenyl)piperazin-1- yl)(1-ethyl-3-methyl-1H-pyrazol-5- yl)methanone | 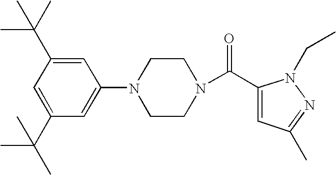 |
| 34 | (5-tert-butylthiophen-2-yl)(4-(3,5-di- tert-butylphenyl)piperazin-1- yl)methanone | 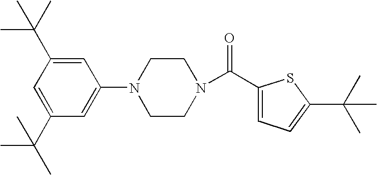 |
| 35 | (5-tert-butyl-2-methylfuran-3-yl)(4- (3,5-di-tert-butylphenyl)piperazin-1- yl)methanone | 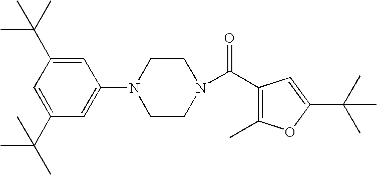 |
| 36 | (4-(3,5-di-tert-butylphenyl)piperazin-1- yl)(4-(1,1,1,3,3,3-hexafluoro-2- hydroxypropan-2-yl)phenyl)methanone | 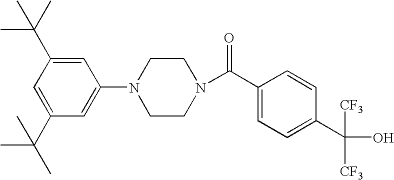 |
| 37 | (4-(3,5-di-tert-butylphenyl)piperazin-1- yl)(2-methyl-5-(trifluoromethyl)oxazol- 4-yl)methanone | 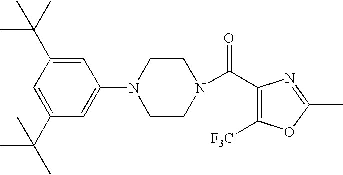 |
| 38 | (4-(3,5-di-tert-butylphenyl)piperazin-1- yl)(2-ethoxypyridin-3-yl)methanone | 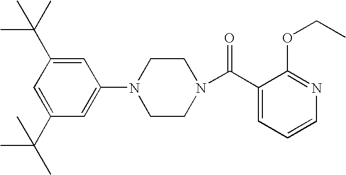 |
| 39 | (4-(3,5-di-tert-butylphenyl)piperazin-1- yl)(5- (trifluoromethyl)benzo[b]thiophen-2- yl)methanone | 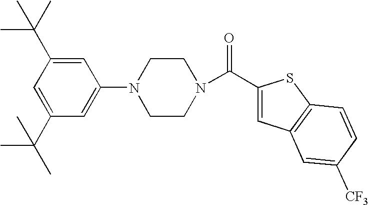 |
| 40 | (2-cyclopropylquinolin-4-yl)(4-(3,5-di- tert-butylphenyl)piperazin-1- yl)methanone | 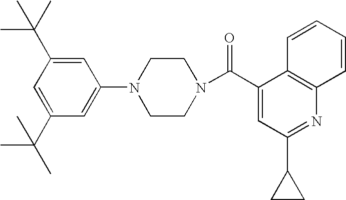 |
| 41 | (4-(3,5-di-tert-butylphenyl)piperazin-1- yl)(2-methyl-4-(trifluoromethyl)thiazol- 5-yl)methanone | 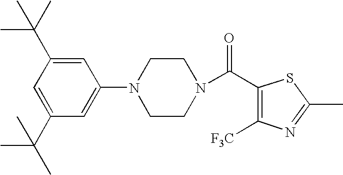 |
| 42 | (3-cyclopropyl-1H-pyrazol-5-yl)(4- (3,5-di-tert-butylphenyl)piperazin-1- yl)methanone | 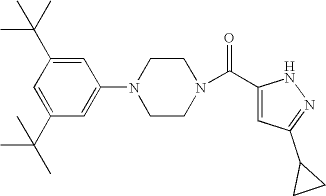 |
| 43 | (4-(3,5-di-tert-butylphenyl)piperazin-1- yl)(5-methyl-4,5,6,7- tetrahydrobenzo[b]thiophen-2- yl)methanone | 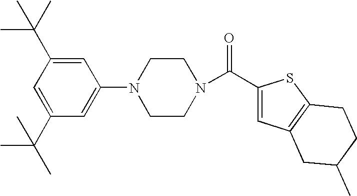 |
| 44 | (4-(3,5-di-tert-butylphenyl)piperazin-1- yl)(5-methyl-4-propylthiophen-2- yl)methanone | 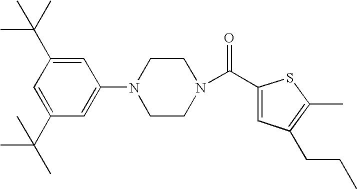 |
| 45 | (4-(3,5-di-tert-butylphenyl)piperazin-1- yl)(2-(ethylthio)pyridin-3-yl)methanone | 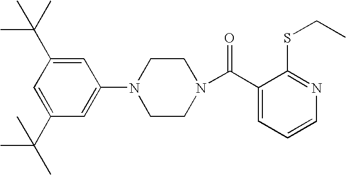 |
| 46 | (4-(3,5-di-tert-butylphenyl)piperazin-1- yl)(4,5-dichloroisothiazol-3- yl)methanone | 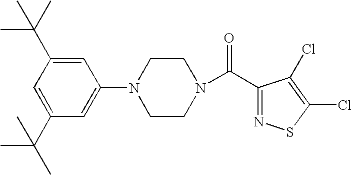 |
| 47 | (4-(3,5-di-tert-butylphenyol)piperazin-1- yl)(6-(2,2,2-trifluoroethoxy)pyridin-3- yl)methanone | 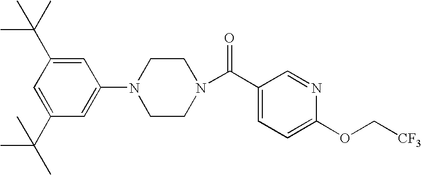 |
| 48 | (4-(3,5-di-tert-butylphenyl)piperazin-1- yl)(phenyl)methanone | 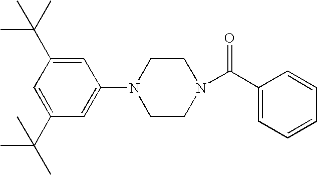 |
| 49 | (4-(3,5-di-tert-butylphenyl)piperazin-1- yl)(6-methylpyridin-2-yl)methanone | 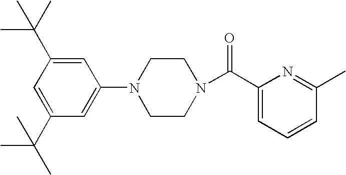 |
| 50 | (4-(3,5-di-tert-butylphenyl)piperazin-1- yl)(2,6-diphenylpyridin-4-yl)methanone | 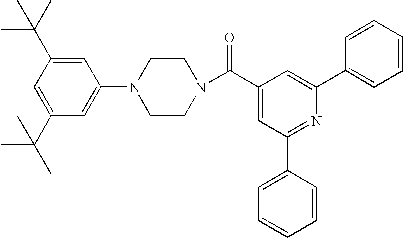 |
| 51 | (4-(3,5-di-tert-butylphenyl)piperazin-1- yl)(5-ethoxyfuran-2-yl)methanone | 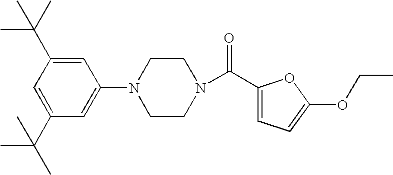 |
| 52 | (4-(3,5-di-tert-butylphenyl)piperazin-1- yl)(4-ethyl-5-methylthiophen-2- yl)methanone | 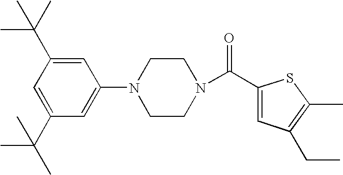 |

### Example 4

### Fluorescent Assay for Cav2.2 Channels Using Potassium Depolarization to Initiate Channel Opening

Human Cav2.2 channels were stably expressed in HEK293 cells along with alpha2-delta and beta subunits of voltage-gated calcium channels. An inwardly rectifying potassium channel (Kir2.3) was also expressed in these cells to allow more precise control of the cell membrane potential by extracellular potassium concentration. At low bath potassium concentration, the membrane potential is relatively negative, and is depolarized as the bath potassium concentration is raised. In this way, the bath potassium concentration can be used to regulate the voltage-dependent conformations of the channels. Compounds are incubated with cells in the presence of low (4 mM) potassium or elevated (12, 25 or 30 mM) potassium to determine the affinity for compound block of resting (closed) channels at 4 mM potassium or affinity for block of open and inactivated channels at 12, 25 or 30 mM potassium. After the incubation period, Cav2.2 channel opening is triggered by addition of higher concentration of potassium (70 mM final concentration) to further depolarize the cell. The degree of state-dependent block can be estimated from the inhibitory potency of compounds after incubation in different potassium concentrations.

Calcium influx through Cav2.2 channels is determined using a calcium sensitive fluorescent dye in combination with a fluorescent plate reader. Fluorescent changes were measured with either a VIPR (Aurora Instruments) or FLIPR (Molecular Devices) plate reader.

Protocol
1. Seed cells in Poly-D-Lysine Coated 96 or 384-well plate and keep in a 37° C.-10% CO₂ incubator overnight
2. Remove media, wash cells with 0.2 mL (96-well plate) or 0.05 mL (384-well plate) Dulbecco's Phosphate Buffered Saline (D-PBS) with calcium & magnesium (Invitrogen; 14040)
3. Add 0.1 mL (96-well plate) or 0.05 mL (384-well plate) of 4 μM fluor-4 (Molecular Probes; F-14202) and 0.02% Pluronic acid (Molecular Probes; P-3000) prepared in D-PBS with calcium & magnesium (Invitrogen; 14040) supplemented with 10 mM Glucose & 10 mM Hepes/NaOH; pH 7.4
4. Incubate in the dark at 25° C. for 60-70 min
5. Remove dye, wash cells with 0.1 mL (96-well plate) or 0.06 mL (384-well plate) of 4, 12, 25, or 30 mM Potassium Pre-polarization Buffer (PPB)
6. Add 0.1 mL (96-well plate) or 0.03 mL (384-well plate) of 4, 12, 25, 30 mM PPB with or without test compound
7. Incubate in the dark at 25° C. for 30 min
8. Read cell plate on VIPR instrument, Excitation=480 nm, Emission=535 nm
9. With VIPR continuously reading, add 0.1 mL (96-well plate) or 0.03 mL (384-well plate) of Depolarization Buffer (DB), which is 2× the final assay concentration, to the cell plate.

| | | | | | | | | | |
|---|---|---|---|---|---|---|---|---|---|
| 4 | mM PPB | 12 | mM PPB | 25 | mM PPB | 30 | mM PPB | 140 | mM K DB |
| 146 | mM NaCl | 138 | mM NaCl | 125 | mM NaCl | 120 | mM NaCl | 10 | mM NaCl |
| 4 | mM KCl | 12 | mM KCl | 25 | mM KCl | 30 | mM KCl | 140 | mM KCl |
| 0.8 | mM CaCl₂ | 0.8 | mM CaCl₂ | 0.8 | mM CaCl₂ | 0.8 | mM CaCl₂ | 0.8 | mM CaCl₂ |
| 1.7 | MgCl₂ | 1.7 | MgCl₂ | 1.7 | MgCl₂ | 1.7 | MgCl₂ | 1.7 | MgCl₂ |
| 10 | HEPES | 10 | HEPES | 10 | HEPES | 10 | HEPES | 10 | HEPES |

| | | | | |
|---|---|---|---|---|
| pH = 7.2 | pH = 7.2 | pH = 7.2 | pH = 7.2 | pH = 7.2 |

### Example 5

### Electrophysiological Measurement of Block of Cav2.2 Channels Using Automated Electrophysiology Instruments

Block of N-type calcium channels is evaluated utilizing the IonWorks HT 384 well automated patch clamp electrophysiology device. This instrument allows synchronous recording from 384 well (48 at a time). A single whole cell recording is made in each well. Whole cell recording is established by perfusion of the internal compartment with amphotericin B.

The voltage protocol is designed to detect use-dependent block. A 2 Hz train of depolarizations (twenty 25 ms steps to +20 mV). The experimental sequence consists of a control train (pre-compound), incubation of cells with compound for 5 minutes, followed by a second train (post-compound). Use dependent block by compounds is estimated by comparing fractional block of the first pulse in the train to block of the 20^{th} pulse.

Protocol:

Parallel patch clamp electrophysiology is performed using IonWorks HT (Molecular Devices Corp) essentially as described by Kiss and colleagues (Kiss et al. 2003; Assay and Drug Development Technologies, 1:127-135). Briefly, a stable HEK 293 cell line (referred to as CBK) expressing the N-type calcium channel subunits (α_{1B}, α₂δ, β₃ₐ) and an inwardly rectifying potassium channel (Kᵢᵣ2.3) is used to record barium current through the N-type calcium channel. Cells are grown in T75 culture plates to 60-90% confluence before use. Cells are rinsed 3× with 10 mL PBS (Ca/Mg-free) followed by addition of 1.0 mL 1× trypsin to the flask. Cells are incubated at 37° C. until rounded and free from plate (usually 1-3 min). Cells are then transferred to a 15 mL conical tube with 13 ml of CBK media containing serum and antibiotics and spun at setting 2 on a table top centrifuge for 2 min. The supernatant is poured off and the pellet of cells is resuspended in external solution (in mM): 120 NaCl, 20 BaCl₂, 4.5 KCl, 0.5 MgCl₂, 10 HEPES, 10 Glucose, pH 7.4). The concentration of cells in suspension is adjusted to achieve 1000-3000 cells per well. Cells are used immediately once they have been resuspended. The internal solution is (in mM): 100 K-Gluconate, 40 KCl, 3.2 MgCl₂, 3 EGTA, 5 HEPES, pH 7.3 with KOH. Perforated patch whole cell recording is achieved by adding the perforating agent amphotericin B to the internal solution. A 36 mg/mL stock of amphtericin B is made fresh in DMSO for each run. 166 μL of this stock is added to 50 mL of internal solution yielding a final working solution of 120 μg/mL.

Voltage protocols and the recording of membrane currents are performed using the IonWorks HT software/hardware system. Currents are sampled at 1.25 kHz and leakage subtraction is performed using a 10 mV step from the holding potential and assuming a linear leak conductance. No correction for liquid junction potentials is employed. Cells are voltage clamped at −70 mV for 10 s followed by a 20 pulse train of 25 ms steps to +20 mV at 2 Hz. After a control train, the cells are incubated with compound for 5 minutes and a second train is applied. Use dependent block by compounds is estimated by comparing fractional block of the first pulse to block of the 20^{th} pulse. Wells with seal resistances less than 70 MOhms or less than 0.1 nA of Ba current at the test potential (+20 mV) are excluded from analysis. Current amplitudes are calculated with the IonWorks software. Relative current, percent inhibition and IC₅₀s are calculated with a custom Excel/Sigmaplot macro.

Compounds are added to cells with a fluidics head from a 96-well compound plate. To compensate for the dilution of compound during addition, the compound plate concentration is 3× higher than the final concentration on the patch plate.

Two types of experiments are generally performed: screens and titrations. In the screening mode, 10-20 compounds are evaluated at a single concentration (usually 3 μM). The percent inhibition is calculated from the ratio of the current amplitude in the presence and absence of compound, normalized to the ratio in vehicle control wells. For generation of IC₅₀s, a 10-point titration is performed on 2-4 compounds per patch plate. The range of concentrations tested is generally 0.001 to 20 μM. IC₅₀s are calculated from the fits of the Hill equation to the data. The form of the Hill equation used is: Relative Current=(Max−Min)/((1+(conc/IC50)̂slope)+Min). Vehicle controls (DMSO) and 0.3 mM CdCl₂ (which inhibits the channel completely) are run on each plate for normalization purposes and to define the Max and Min.

### Example 6

### Electrophysiological Measurement of Block of Cav2.2 Channel Using Whole Cell Voltage Clamp and Using PatchXpress Automated Electrophysiology Instrument

Block of N-type calcium channels is evaluated utilizing manual and automated (PatchXpress) patch clam electrophysiology. Voltage protocols are designed to detect state-dependent block. Pulses (50 ms) are applied at a slow frequency (0.067 Hz) from polarized (−90 mV) or depolarized (−40 mV) holding potentials. Compounds which preferentially block inactivated/open channels over resting channels will have higher potency at −40 mV compared to −90 mV.

Protocol:

A stable HEK 293 cell line (referred to as CBK) expressing the N-type calcium channel subunits (α_{1B}, α₂δ, β₃ₐ) and an inwardly rectifying potassium channel (Kᵢᵣ2.3) is used to record barium current through the N-type calcium channel. Cells are grown either on poly-D-lysine coated coverglass (manual EP) or in T75 culture plates (PatchXpress). For the PatchExpress, cells are released from the flask using trypsin. In both cases, the external solution is (in mM): 130 CsCl₂, 10 EGTA, 10 HEPES, 2 MgCl₂, 3 MgATP, pH 7.3 with CsOH.

Barium currents are measured by manual whole-cell patch clamp using standard techniques (Hamill et. Al. Pfluegers Archiv 391:85-100 (1981)). Microelectrodes are fabricated from borosilicate glass and fire-polished. Electrode resistances are generally 2 to 4 MOhm when filled with the standard internal saline. The reference electrode is a silver-silver chloride pellet. Voltages are not corrected for the liquid junction potential between the internal and external solutions and leak is subtracted using the P/n procedure. Solutions are applied to cells by bath perfusion via gravity. The experimental chamber volume is ˜0.2 mL and the perfusion rate is 0.5-2 mL/min. Flow of suction through the chamber is maintained at all times. Measurement of current amplitudes is performed with PULSEFIT software (HEKA Elektronik).

PatchXpress (Molecular Devices is a 16-well whole-cell automated patch clamp device that operates asynchronously with fully integrated fluidics. High resistance (gigaohm) seals are achieved with 50-80% success. Capacitance and series resistance compensation is automated. No correction for liquid junction potentials is employed. Leak is subtracted using the P/n procedure. Compounds are added to cells with a pipettor from a 96-well compound plate. Voltage protocols and the recording of membrane currents are performed using the PatchXpress software/hardware system. Current amplitudes are calculated with DataXpress software.

In both manual and automated patch clamp, cells are voltage clamped at −4 mV or −90 mV and 50 ms pulses to +20 mV are applied every 15 sec (0.067 Hz). Compounds are added in escalating doses to measure % inhibition. Percent inhibition is calculated from the ratio of the current amplitude in the presence and absence of compound. When multiple doses are achieved per cell, IC₅₀s are calculated. The range of concentrations tested is generally 0.1 to 30 μM. IC₅₀s are calculated from the fits of the Hill equation to the data. The form of the Hill equation used is:

Relative Current=1/(1+(conc/IC₅₀)̂slope).

### Example 7

### Assay for Cav3.1 and Cav3.2 Channels

The T-type calcium channel blocking activity of the compounds of this invention may be readily determined using the methodology well known in the art described by Xia, et al., Assay and Drug Development Tech., 1 (5), 637-645 (2003).

In a typical experiment ion channel function from HEK 293 cells expressing the T-type channel alpha-1G, H, or I (CaV 3.1, 3.2, 3.3) is recorded to determine the activity of compounds in blocking the calcium current mediated by the T-type channel alpha-1G, H, or I (CaV 3.1, 3.2, 3.3). In this T-type calcium (Ca²⁺) antagonist voltage-clamp assay calcium currents are elicited from the resting state of the human alpha-1G, H, or I (CaV 3.1, 3.2, 3.3) calcium channel as follows. Sequence information for T-type (Low-voltage activated) calcium channels are fully disclosed in e.g., U.S. Pat. No. 5,618,720, U.S. Pat. No. 5,686,241, U.S. Pat. No. 5,710,250,U.S. Pat. No. 5,726,035, U.S. Pat. No. 5,792,846, U.S. Pat. No. 5,846,757, U.S. Pat. No. 5,851,824, U.S. Pat. No. 5,874,236, U.S. Pat. No. 5,876,958, U.S. Pat. No. 6,013,474, U.S. Pat. No. 6,057,114, U.S. Pat. No. 6,096,514, WO 99/28342, and J. Neuroscience, 19 (6):1912-1921 (1999). Cells expressing the t-type channels were grown in H3D5 growth media which is comprised DMEM, 6% bovine calf serum (HYCLONE), 30 micromolar Verapamil, 200 microgram/ml Hygromycin B, 1× Penicillin/Streptomycin. Glass pipettes are pulled to a tip diameter of 1-2 micrometer on a pipette puller. The pipettes are filled with the intracellular solution and a chloridized silver wire is inserted along its length, which is then connected to the headstage of the voltage-clamp amplifier. Trypsinization buffer was 0.05% Trypsin, 0.53 mM EDTA. The extracellular recording solution consists of (mM): 130 mM NaCl, 4 mM KCl, 1 mM MgCl2, 2 mM CaCl2, 10 mM HEPES, 30 Glucose, pH 7.4. The internal solution consists of (mM): 135 mM CsMeSO4, 1 MgCl2, 10 CsCl, 5 EGTA, 10 HEPES, pH 7.4, or 135 mM CsCl, 2 MgCl2, 3 MgATP, 2 Na2ATP, 1 Na2GTP, 5 EGTA, 10 HEPES, pH 7.4. Upon insertion of the pipette tip into the bath, the series resistance is noted (acceptable range is between 1-4 megaohm). The junction potential between the pipette and bath solutions is zeroed on the amplifier. The cell is then patched, the patch broken, and, after compensation for series resistance (>=80%), the voltage protocol is applied while recording the whole cell Ca2+ current response. Voltage protocols: (1) −80 mV holding potential every 20 seconds pulse to −20 mV for 40 msec duration; the effectiveness of the drug in inhibiting the current mediated by the channel is measured directly from measuring the reduction in peak current amplitude initiated by the voltage shift from −80 mV to −20 mV; (2). −100 mV holding potential every 15 seconds pulse to −20 mV for 40 msec duration; the effectiveness of the drug in inhibiting the current mediated by the channel is measured directly from measuring the reduction in peak current amplitude initiated by the shift in potential from −100 mV to −30 mV. The difference in block at the two holding potentials was used to determine the effect of drug at differing levels of inactivation induced by the level of resting state potential of the cells. After obtaining control baseline calcium currents, extracellular solutions containing increasing concentrations of a test compound are washed on. Once steady state inhibition at a given compound concentration is reached, a higher concentration of compound is applied. % inhibition of the peak inward control Ca2+ current during the depolarizing step to −20 mV is plotted as a function of compound concentration.

The intrinsic T-type calcium channel antagonist activity of a compound which may be used in the present invention may be determined by these assays.

### Example 8

### In Vitro Results

TABLE 2

| | | Cav 2.2 | Cav 2.2 | Cav 3.1 | Cav 3.2 |
|---|---|---|---|---|---|
| | Cav 2.2 | % Inh @ | IC50 | IC50 | IC50 |
| Compound | % Inh @ 30 μM | 10 μM | (μM) | (μM) | (μM) |
| 1 | 101 | 91 | | | |
| 2 | 94 | 90 | | | |
| 4 | 97 | 92 | 2.21 | 0.89 | 0.79 |
| 5 | 96 | 85 | | | |
| 6 | 95 | 86 | | | |
| 7 | 96 | 97 | 0.30 | 0.47 | 0.66 |
| 8 | 95 | 94 | 0.65 | 0.55 | 0.71 |
| 9 | 101 | 91 | | | |
| 10 | 104 | 96 | 0.37 | 1.07 | 1.31 |
| 11 | 97 | 91 | | | |
| 12 | 98 | 84 | | | |
| 13 | 97 | 97 | 0.62 | 0.60 | 0.68 |
| 14 | 93 | 86 | 1.37 | 1.28 | 1.11 |
| 15 | 93 | 96 | | | |
| 16 | 90 | 96 | 1.46 | 0.68 | 0.69 |
| 17 | 97 | 80 | | | |
| 18 | 83 | 59 | | | |
| 19 | 69 | 49 | | | |
| 21 | 78 | 53 | | | |
| 22 | 103 | 99 | 0.99 | 0.05 | 0.07 |
| 23 | 97 | 90 | 1.40 | 0.80 | 0.87 |
| 24 | 96 | 88 | | | |
| 25 | 99 | 89 | | | |
| 26 | 98 | 87 | | | |
| 27 | 83 | 59 | | | |
| 28 | 101 | 87 | 2.40 | 0.80 | 0.96 |
| 29 | 101 | 88 | 2.00 | 0.84 | 1.01 |
| 30 | 100 | 91 | 1.30 | 0.97 | 1.15 |
| 31 | 104 | 99 | 0.85 | 1.25 | 1.59 |
| 32 | 98 | 98 | 0.78 | 1.09 | 1.15 |
| 33 | 95 | 97 | 0.83 | 2.34 | 2.52 |
| 34 | 59 | 52 | | | |
| 35 | 95 | 77 | | | |
| 36 | 73 | 32 | | | |
| 37 | 101 | 94 | 0.55 | 2.04 | 2.29 |
| 38 | 98 | 107 | 0.85 | 1.49 | 2.23 |
| 39 | 41 | 15 | | | |
| 40 | 91 | 86 | | | |
| 41 | 107 | 102 | 0.83 | 1.51 | 1.41 |
| 42 | 98 | 54 | | | |
| 43 | 57 | 46 | | | |
| 44 | 81 | 65 | | | |
| 45 | 101 | 97 | 1.08 | 0.96 | 1.80 |
| 46 | 89 | 79 | | | |
| 47 | 104 | 101 | 1.48 | 0.49 | 0.80 |
| 48 | 103 | 103 | 1.48 | 0.37 | 0.61 |
| 49 | 105 | 102 | 1.66 | 1.52 | 1.88 |
| 50 | 26 | 20 | | | |
| 51 | 100 | 98 | | | |
| 52 | 88 | 75 | | | |

### Example 9

### In Vivo Assay 1

### Rodent CFA Model

Male Sprague Dawley rates (300-400 g) are administered 200 μL CFA (complete Freund's Adjuvant) three days prior to the study. CFA is mycobacterium tuberculosis suspended in saline (1:1; Sigma) to form an emulsion that contains 0.5 mg mycobacterium/mL. The CFA is injected into the plantar area of the left hind paw.

Rats are fasted the night before the study only for oral administration of the compounds. On the morning of the test day using a Ugo Basile apparatus, 2 baseline samples can be taken 1 hour apart. The rat is wrapped in a towel. Its paw is placed over a ball bearing and under the pressure device. A foot pedal is depressed to apply constant linear pressure. Pressure is stopped when the rat withdraws its paw, vocalizes, or struggles. The right paw is then tested. Rats can then be dosed with compound and tested at predetermined time points.

Compounds are prepared in DMSO (15%)/PEG300 (60%)/Water (25%) and were dosed in a volume of 2 mL/kg.

Percent maximal possible effect (% MPE) can be calculated as: (post-treatment−pre-treatment)/(pre-injury threshold−pre-treatment)×100. The % responder is the number of rats that have an MPE 30% at any time following compound administration. The effect of treatment can be determined by one-way ANOVA Repeated Measures Friedman Test with a Dunn's post test.

### Example 10

### In Vivo Assay 2

### Formalin-Induced Pain Model

The effects of intrathecally delivered compounds of the invention on the rat formalin model can also be measured. The compounds can be reconstituted to stock solutions of approximately 10 mg/mL in propylene glycol. Typically eight Holtzman male rats of 275-375 g size are randomly selected per test article.

The following study groups can be used, with test article, vehicle control (propylene glycol) and saline delivered intraperitoneally (IP):

Formalin Model Dose Groups

| Test/Control Article | Dose | Route | Rats per group |
|---|---|---|---|
| Compound | 30 mg/kg | IP | 6 |
| Propylene glycol | N/A | IP | 4 |
| Saline | N/A | IP | 7 |

N/A = Not Applicable

Prior to initiation of drug delivery baseline behavioral and testing data can be taken. At selected times after infusion of the Test or Control Article these data can then be again collected.

On the morning of testing, a small metal band (0.5 g) is loosely placed around the right hind paw. The rat is placed in a cylindrical Plexiglas chamber for adaptation a minimum of 30 minutes. Test Article or Vehicle Control Article is administered 10 minutes prior to formalin injection (50 μL of 5% formalin) into the dorsal surface of the right hindpaw of the rat. The animal is then placed into the chamber of the automated formalin apparatus where movement of the formalin injected paw is monitored and the number of paw flinches tallied by minute over the next 60 minutes (Malmberg, A. B., et al., Anesthesiology (1993) 79:270 281).

Results can be presented as Maximum Possible Effect ±SEM, where saline control=100%.

### Example 11

### In Vivo Assay 3

### Spinal Nerve Ligation Model of Neuropathic Pain

Spinal nerve ligation (SNL) injury can be induced using the procedure of Kim and Chung, (Kim, S. H., et al., Pain (1992) 50:355-363) in male Sprague-Dawley rats (Harlan; Indianapolis, Ind.) weighing 200 to 300 grams. Anesthesia is induced with 2% halothane in O₂ at 2 L/min and maintained with 0.5% halothane in O₂. After surgical preparation of the rats and exposure of the dorsal vertebral column from L4 to S2, the L5 and L6 spinal nerves are tightly ligated distal to the dorsal root ganglion using 4-0 silk suture. The incision is closed, and the animals are allowed to recover for 5 days. Rats that exhibit motor deficiency (such as paw-dragging) or failure to exhibit subsequent tactile allodynia are excluded from further testing. Sham control rats undergo the same operation and handling as the experimental animals, but without SNL.

The assessment of tactile allodynia consists of measuring the withdrawal threshold of the paw ipsilateral to the site of nerve injury in response to probing with a series of calibrated von Frey filaments. Each filament is applied perpendicularly to the plantar surface of the ligated paw of rats kept in suspended wire-mesh cages. Measurements are taken before and after administration of drug or vehicle. Withdrawal threshold is determined by sequentially increasing and decreasing the stimulus strength (“up and down” method), analyzed using a Dixon non-parametric test (Chaplan S. R., et al., J Pharmacol Exp Ther (1994) 269:1117-1123), and expressed as the mean withdrawal threshold.

The method of Hargreaves and colleagues (Hargreaves, K., et al., Pain (1988) 32:77-8) can be employed to assess paw-withdrawal latency to a thermal nociceptive stimulus. Rats are allowed to acclimate within a plexiglass enclosure on a clear glass plate maintained at 30° C. A radiant heat source (i.e., high intensity projector lamp) is then activated with a timer and focused onto the plantar surface of the affected paw of nerve-injured or carrageenan-injected rats. Paw-withdrawal latency can be determined by a photocell that halts both lamp and timer when the paw is withdrawn. The latency to withdrawal of the paw from the radiant heat source is determined prior to carrageenan or L5/L5 SNL, 3 hours after carrageenan or 7-21 days after L5/L6 SNL but before drug and after drug administration. A maximal cut-off of 40 seconds is employed to prevent tissue damage. Paw withdrawal latencies can be thus determined to the nearest 0.1 second. Reversal of thermal hyperalgesia is indicated by a return of the paw withdrawal latencies to the pre-treatment baseline latencies (i.e., 21 seconds). Anti-nociception is indicated by a significant (p<0.05) increase in paw withdrawal latency above this baseline. Data is converted to % anti hyperalgesia or % anti-nociception by the formula: (100×(test latency−baseline latency)/(cut-off−baseline latency) where cut-off is 21 seconds for determining anti-hyperalgesia and 40 seconds for determining anti-nociception.

1. A method to treat a condition modulated by calcium ion channel activity, which method comprises administering to a subject in need of such treatment an amount of the compound of formula (1) effective to ameliorate said condition, wherein said compound is of the formula: 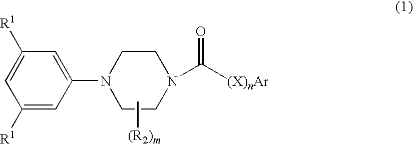
or a pharmaceutically acceptable salt or conjugate thereof, wherein
R₁ are independently C(CH₃)₃ or C(CF₃)₃;
each R₂ is independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′, —C(CH₃)₂CONR′₂, ═O, and ═NOR′, wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or R₂ may be one or more optionally substituted groups selected from alkyl (1-6C), alkenyl (2-6C), alkynyl (2-6C), heteroalkyl (2-6C), heteroalkenyl (2-6C), heteroalkynyl (2-6C);
m is 0-4 and n is 0-1;
X is alkylenyl (1-3C) or heteroalkylenyl (1-3C);
Ar is an optionally substituted aryl (6-10C) or heteroaryl (5-12 ring members);
wherein the optional substituents on Ar are independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′ and —C(CH₃)₂CONR′₂ wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12 ring members).

2. The method of claim 1 wherein said condition is chronic or acute pain, a mood disorder, a neurodegenerative disorder, a hearing disorder, a gastrointestinal disorder, a genitorurinary disorder, neuroprotection, a metabolic disorder, cardiovascular disease, epilepsy, diabetes, cancer, a sleep disorder, Parkinson's disease, schizophrenia or male birth control.

3. The method of claim 1 wherein said condition is chronic or acute pain.

4. The method of claim 1 wherein R₁ is C(CH₃)₃.

5. The method of claim 1 wherein m is 0-2.

6. The method of claim 1 wherein m is 0.

7. The method of claim 1 wherein n is 0.

8. The method of claim 1 wherein Ar is an optionally substituted furanyl, pyridinyl, thiadizolyl, phenyl, thiophenyl, pyrazolyl, thiazolyl, napthyl, naphthyridinyl, imidazolyl, isoxazolyl, pyrazolopyridinyl, oxazolyl, benzothiophenyl, quinolinyl or isothiazolyl.

9. A pharmaceutical composition comprising a compound of the formula: 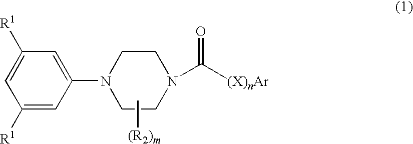
or a pharmaceutically acceptable salt or conjugate thereof, in admixture with a pharmaceutically acceptable excipient, wherein
R₁ are independently C(CH₃)₃ or C(CF₃)₃;
each R₂ is independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′, —C(CH₃)₂CONR′₂, ═O, and ═NOR′, wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or R₂ may be one or more optionally substituted groups selected from alkyl (1-6C), alkenyl (2-6C), alkynyl (2-6C), heteroalkyl (2-6C), heteroalkenyl (2-6C), heteroalkynyl (2-6C);
m is 0-4 and n is 0-1;
X is alkylenyl (1-3C) or heteroalkylenyl (1-3C);
Ar is an optionally substituted aryl (6-10C) or heteroaryl (5-12 ring members);
wherein the optional substituents on Ar are independently selected from halo, CN, NO₂, CF₃, OCF₃, COOR′, CONR′₂, OR′, SR′, SOR′, SO₂R′, NR′₂, NR′(CO)R′, NR′SO₂R′, —Si(CH₃)₃, —CH₂CN, —C(CH₃)₂CN, —C(CH₃)₂CH₂OR′, —C(CH₃)₂CO₂R′, —C(CH₃)₂CONHR′ and —C(CH₃)₂CONR′₂ wherein each R′ is independently H or an optionally substituted group selected from alkyl (1-6C), alkenyl (2-6C), alkynyl (2-6C), heteroalkyl (2-6C), heteroalkenyl (2-6C), heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), heteroalkynyl (2-6C), aryl (6-10C), heteroaryl (5-12 ring members).

10. The pharmaceutical composition of claim 9 wherein R₁ is C(CH₃)₃.

11. The pharmaceutical composition of claim 9 wherein m is 0-2.

12. The pharmaceutical composition of claim 9 wherein m is 0.

13. The pharmaceutical composition of claim 9 wherein n is 0.

14. The pharmaceutical composition of claim 9 wherein Ar is an optionally substituted furanyl, pyridinyl, thiadizolyl, phenyl, thiophenyl, pyrazolyl, thiazolyl, napthyl, naphthyridinyl, imidazolyl, isoxazolyl, pyrazolopyridinyl, oxazolyl, benzothiophenyl, quinolinyl or isothiazolyl.
